(19) United States
(12) Patent Application Publication
(10) Pub. No.: US 2023/0057155 A1
(43) Pub. Date: Feb. 23, 2023

(54) DRY LIPOSOME FORMULATIONS AND RELATED METHODS THEREOF

(71) Applicant: BOARD OF REGENTS, THE UNIVERSITY OF TEXAS SYSTEM, Austin, TX (US)

(72) Inventors: Zhengrong CUI, Austin, TX (US); Robert O. WILLIAMS, III, Austin, TX (US); Khaled ABOULFOTOUH, Austin, TX (US)

(73) Assignee: BOARD OF REGENTS, THE UNIVERSITY OF TEXAS SYSTEM, Austin, TX (US)

(21) Appl. No.: 17/819,268

(22) Filed: Aug. 11, 2022

Related U.S. Application Data
(60) Provisional application No. 63/232,099, filed on Aug. 11, 2021.

(51) Int. Cl. A61K 9/127; A61K 45/06; A61K 47/26

(52) U.S. Cl. CPC A61K 9/1271; A61K 9/1277; A61K 45/06; A61K 47/26

(57) ABSTRACT

Described herein are dry powder compositions of liposomes. Formulations containing a cryoprotectant can be converted to dry powders using, e.g., thin-film freeze-drying (TFFD) to obtain stabilized composition that show improved properties.

This application claims the benefit of priority to U.S. Provisional Application No. 63/232,099, filed on Aug. 11, 2021, the entire contents of which are hereby incorporated by reference.

### BACKGROUND

### I. Field

The disclosure generally relates to dry liposomes preparations. More particularly, the disclosure relates to dry thin film compositions containing liposomes and pharmaceutical formulations produced from aqueous liposome-containing compositions.

### II. Related Art

Liposomes are spherical vesicles consisting of one or more phospholipid bilayers surrounding an aqueous core. The liposome diameter ranges from 20 nm to 10 µm. The liposomal vesicles are usually made of neutral and synthetic lipids together with cholesterol (Drulis-Kawa and Dorotkiewicz-Jach, 2010). Liposomes are universal drug carriers (i.e., can encapsulate both hydrophilic and hydrophobic drugs) in the cosmetic and pharmaceutical industries. They can be tailored to achieve desired pharmacokinetic and pharmacodynamic characteristics. Liposomes can also enhance the drug localization in the disease site (e.g., tumor tissues) (Barenholz, 2001). Thus, they have been intensively investigated as drug carriers and vaccine adjuvants during the past 50 years (Ingvarsson et al., 2011). The efforts of pharmaceutical scientists in optimizing the liposomal formulations (e.g., by enhancing their stability) have resulted in the FDA approval of many liposome-based products (e.g., Doxil®, Amphotec® and AmBisome®) for intravenous administration (Barenholz, 2001).

However, the development of liposomal formulations is challenged by lipid oxidation and/or hydrolysis, drug leakage and aggregation or fusion of vesicle membranes. An efficient strategy to overcome these challenges is to convert the liposome dispersions into dry powders by various techniques (Franze et al., 2018). Freeze-drying is the most investigated technique to formulate dry liposome formulations (Ingvarsson et al., 2011). However, the optimization of the freeze-drying conditions is still challenging after decades of studies. The slow freezing rates of conventional freeze-drying process often result in the formation of large ice crystals and thick ice channels. The latter allow sufficient time for liposome particles to aggregate and grow (Engstrom et al., 2008). Additionally, slow freezing can greatly affect the liposomal membrane integrity. For example, the ice-liquid interface and phase separation result in liposome concentration and thus aggregation and disruption of the phospholipid bilayer structure (Ingvarsson et al., 2011). Damage of liposomal membrane integrity leads to leakage of encapsulated drug (Yu et al., 2020). On the other hand, fast freezing rate induces the formation of small ice crystals and thus homogeneous distribution of the cryoprotectant(s) which in turn diminish the disruption of the liposomal phospholipid bilayer structure. Fast freezing rates can be achieved by different techniques including thin-film freeze-drying (TFFD) and spray freeze-drying (SFD) technologies (Engstrom et al., 2008). TFFD process involves applying of the liquid formulation onto a cryogenically cooled surface to form frozen thin-films which are subsequently lyophilized (AboulFotouh et al., 2020). Compared to SFD, TFFD process is advantageous in terms of small surface area of the gas-liquid interface and the absence of strong shear stress associated with SFD that can affect the liposomal membrane integrity as well as the integrity of loaded cargos (e.g., protein therapeutics and nucleic acids) (Zhang et al., 2021; Hufnagel et al., 2022; AboulFotouh et al., 2021a). Importantly, TFFD-processed powders are often porous with brittle matrices, low densities and large surface areas (Wang et al., 2014). Thus, TFFD-powders with the proper compositions and processed at certain freezing and drying conditions are inhalable and can reach deep lung (Sahakijpijarn et al., 2020; Sahakijpijarn et al., 2021). This can solve a great problem encountered by the pharmaceutical industry, namely the formulation of inhalable liposome dry powders which is very challenging (Yu et al., 2020).

### SUMMARY

In an aspect the present disclosure provides a dry liposome powder composition wherein said composition comprises a liposomal formulation and a sugar and/or a sugar alcohol. The liposomal formulation may comprise a neutral lipid, a cationic lipid, an amphipathic lipid and/or an anionic lipid. The liposomal formulation may in particular comprise dioleoyl phosphatidylcholine and cholesterol. The composition may have a particle size distribution within 10%-50% of the range of a corresponding liquid liposomal composition upon reconstitution. The sugar or sugar alcohol may be present at a lipid to sugar and/or sugar alcohol ratio of about 1:0.6 to 1:30 w/w respectively in some embodiments and with some liposomal formulations and certain types of compositions or about 1:250 to 1:1000 w/w, respectively in other embodiments and with other liposomal formualtions and certain types of composition. The sugar or sugar alcohol may be mannitol, sucrose and/or trehalose. The composition may comprise less than 5% water.

The composition may further comprise an excipient, such as a salt, a buffer, a detergent, a polymer, an amino acid, a second sugar or a preservative. In particular, the excipient may be disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, agarose, sorbitol, maltose, trehalose, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco’s modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. The composition may comprise about 60-95% w/w of said excipient.

The composition may be prepared from a liquid liposomal formulation. The composition may comprises a lipid selected from the group consisting of for example soya lecithin, cholesterol, Soya phosphatidylcholine, hydrogenated soybean phosphatidylcholine, 1,2-Distearoyl-sn-glycero-3-phosphoglycerol, phosphatidylcholine, dipalmitoylphosphatidylcholine, egg phosphatidylcholine, 1,2-diphytanoyl-sn-glycero-3-phosphocholine, 1,2-Dioleoyl-sn-glycero-3-phospho-rac-(1-glycerol), 1,2-dioleoyl-3-trimethylammonium-propane, 1,2-Dioleoyl-sn-glycero-3-phosphocholine, 1,2-dipalmitoyl-sn-glycero-3-phosphocholine, 1-paltnitoy1-2-1yso-sn-gycero-3-pllosplloclloline, 1-palmitoyl-2-oleoyl-sn-glycero-3-ethylphosphocholine, 1,2-dimyristoyl-sn-glycero-3-phosphocholine, 1-palmitoy1-2-oleoyl-sn-glycero-3-phosphocholine, and 1,2-dioleoyl-sn-glycero-3-phosphoethanolamine. The composition may further comprise a drug or biologic agent, such as an antifungal, an antibacterial, a cancer chemotherapeutical agent, a protein (e.g., enzyme, monoclonal antibody), or nucleic acid.

Also provides is a method for preparing a thin film liposomal composition comprising applying a liquid liposomal composition and a sugar or a sugar alcohol to a freezing surface; and allowing said liquid liposomal composition to disperse and freeze on said freezing surface thereby forming a thin film. The thin film liposome composition may further comprise a drug or biologic agent. The liquid liposomal composition may comprise a neutral lipid, a cationic lipid, an amphipathic lipid and/or an anionic lipid, and in particular may comprise dioleoyl phosphatidylcholine and cholesterol. The thin film may have a particle size distribution upon reconstitution within about 10-50% or about 30% of the range of the liquid liposomal composition. Also provided is a thin film made according to such methods.

The sugar or sugar alcohol may be present at about a lipid to sugar and/or sugar alcohol ratio of about 1:0.6 to 1:30 w/w, respectively or about 1:250 to 1:1000 w/w, respectively. The sugar or sugar alcohol may be mannitol, sucrose and/or trehalose. The thin film may comprise less than about 5% water. The thin film may further comprise an excipient, such as a salt, a buffer, a detergent, a polymer, an amino acid, a second sugar or a preservative. In particular, the excipient may bedisodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, agarose, sorbitol, maltose, trehalose, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco’ s modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. The excipient may comprise from about 60% to about 95% w/w of composition.

The liquid liposomal composition may be exposed to said freezing surface from about 50 milliseconds to about 5 seconds. The exposure may comprise spraying or dripping droplets of said liquid liposomal composition, such as wherein the freezing surface temperature is about -180° C. to about 0° C., the diameters of the droplets are about 2-5 millimeters, and the droplets are dropped from a distance about 2 cm to 10 cm from the freezing surface. The method may further comprise contacting the droplets with a freezing surface having a temperature differential of at least about 30° C. between the droplets and the surface, such as where the freezing rate of said droplets is between about 10 K/second and about 10³ K/second.

The method may further comprise removing the solvent from the thin film to form a dry composition, such as by lyophilization. The method may also further comprise solvating said dry liposomal composition, thereby forming a reconstituted liquid composition. Solvating of said dry liposomal composition may occur at least one year after preparing said dry liposomal composition from said liquid liposomal composition. Prior to said solvating of said dry liposomal composition, said dry liposomal composition may be stored at about 4° C. for at least 99% of the time. Upon solvating said dry liposomal composition the resulting reconstituted liquid liposomal composition may remain homogeneous for at least one week when stored properly, and/or the resulting reconstituted liquid liposomal composition may not form a precipitate for at least one week when stored properly. The liquid liposomal composition may further comprise a pharmacologically active ingredient, such as drug or biologic agent, such as an antifungal, an antibacterial, a cancer chemotherapeutical agent, a protein (e.g., enzyme, monoclonal antibody), or nucleic acid. The thin film liposomal formulation may be formulated as an aerosol/for inhalation.

The liquid liposomal composition may comprise a lipid selected from the group consisting of soya lecithin, cholesterol, Soya phosphatidylcholine, hydrogenated soybean phosphatidylcholine, 1,2-Distearoyl-sn-glycero-3-phosphoglycerol, phosphatidylcholine, dipalmitoylphosphatidylcholine, egg phosphatidylcholine, 1,2-diphytanoyl-sn-glycero-3-phosphocholine, 1,2-Dioleoyl-sn-glycero-3-phospho-rac-(1-glycerol), 1,2-dioleoyl-3-trimethylammonium-propane, 1,2-Dioleoyl-sn-glycero-3-phosphocholine, 1,2-dipalmitoyl-sn-glycero-3-phosphocholine, 1-paltnitoyl-2-lyso-sn-gycero-3-pllosplloclloline, 1-palmitoyl-2-oleoyl-sn-glycero-3-ethylphosphocholine, 1,2-dimyristoyl-sn-glycero-3-phosphocholine, 1-palmitoyl-2-oleoyl-sn-glycero-3-phosphocholine, and 1,2-dioleoyl-sn-glycero-3-phosphoethanolamine. The liquid liposomal composition may in particular comprise a lipid selected from the group consisting of cholesterol and 1,2-dioleoyl-sn-glycero-3-phosphocholine.

In an additional embodiment, there is a method of treating a disease in a subject, said method comprising administering a therapeutically effective amount of a composition as described above to said subject, or a solvated, rehydrated or reconstituted version thereof. Administering may be oral administering, intravenous administering, intra-arterial administering, subcutaneous or other parenteral routes of administration, topical administering, intranasal administering, pulmonary administering into the lungs, or intravaginal administering. In particular, administering may be via inhalation.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Particle size distribution of neutral liposomes before (i.e., liquid dispersions) and after (i.e., dry powders reconstituted in water) TFFD determined by DLS. The liposomes are composed of DOPC and cholesterol at a molar ratio of 1:1.

FIGS. 2A-I. Particle size distribution of anionic liposomes before (i.e., liquid dispersions) and after (i.e., dry powders reconstituted in water) TFFD determined by DLS. The liposomes are composed of DOPA and cholesterol at a molar ratio of 1:1.

FIGS. 3A-I. Particle size distribution of cationic liposomes before (i.e., liquid dispersions) and after (i.e., dry powders reconstituted in water) TFFD determined by DLS. The liposomes are composed of DOTAP and cholesterol at a molar ratio of 1:1.

FIGS. 4A-D. (FIGS. 4A-C) Particle size distribution of AmBisome® after reconstituting the lyophilized powder in 12 mL of sterile water (i.e., before TFFD) and after the dispersion was processed into dry powders using TFFD and reconstituted again (i.e., after TFFD). Particle size distribution was determined by DLS. (A) No additional sucrose was added. (FIGS. 4B and 4C) Sucrose was added to achieve a sucrose to total lipid ratios of 5:1 w/w and 16:1 w/w, respectively. (FIG. 4D) The aerosol performance of TFFD-processed dry powder of original AmBisome® formulation (i.e., no additional excipients were added) determined by a Next Generation Pharmaceutical Impactor.

FIGS. 5A-E. Particle size distribution of Doxil® STEALTH® liposome dispersions before TFFD and after the dispersions were processed into dry powders using TFFD and reconstituted (i.e., after TFFD). Particle size distribution was determined by DLS.

FIGS. 6A-I. Particle size distribution of CpG ODNs-loaded cationic liposome dispersions before TFFD and after the dispersions were processed into dry powders using TFFD and reconstituted (i.e., after TFFD). Particle size distribution was determined by DLS.

### DETAILED DESCRIPTION

Here, the inventors disclose dry powder compositions of liposomal formulations. Formulations containing one or two sugar or sugar alcohol (i.e., sucrose, trehalose and/or mannitol) at lipid to sugar(s)/sugar alcohol weight ratios of 1:0.6, 1:2.5, 1:4, 1:5, 1:8, 1:15, 1:16, 1:30, 1:250, 1:500 and 1:1000 were converted to dry powders using thin-film freeze-drying (TFFD). A sugar/sugar alcohol to lipid weight ratio of 15:1 or 16:1 is usually effective in protecting the liposomal membrane structure against freezing-induced stresses and thus maintaining the liposomes’ mean particle size after TFFD and reconstitution. However, further optimization of the sugar/sugar alcohol to lipid weight/molar ratio may be necessary depending on the liposome membrane structure (i.e., type and weight/molar ratios of different lipids) and the presence of other excipients (e.g., buffers and tonicity adjusters). Additionally, the initial concentration (% w/v) of sugar/sugar alcohol in the liquid liposomal dispersion (i.e., before TFFD) is also crucial because it affects the dispersion’s viscosity. Herein, the sugar/sugar alcohol concentration in the liquid liposomal dispersion was 1, 2, 3, 4, 7, 8 or 25% w/v. In most cases, sucrose was the most effective cryoprotectant in maintaining the liposome membrane integrity followed by trehalose. However, mannitol was found to be as effective as sucrose and even more effective than trehalose as a cryoprotectant depending on the liposome membrane structure.

Liquid liposome dispersions were dropped onto a cryogenically cooled surface to form frozen thin films rapidly. The frozen films were then lyophilized to sublime water. Some dry powders maintained the particle size distribution of the original liquid formulations after they were reconstituted. This solves a major problem associated with the liposomal preparations, namely, damage due to harmful freezing temperatures. In addition, liposomal formulations may be manufactured and distributed as a dry powder in a single vial which may not be adversely affected by cold chain failure or may avoid cold chain requirement. The powder can be reconstituted using a commonly used diluent (e.g., water for injection or normal saline).

These dry powder formulations have a number of distinct advantages. For example, thin film freezing is an ultra-rapid freezing process (i.e., 100-1000 K/s) that can preserve the particle size distribution via accelerating the nucleation rate and the formation of small ice crystals. Other high-speed freezing methods may also be employed. Technologies with slower freezing rate (e.g., conventional shelf freeze-drying) result in phase separation and liposome concentration and thus fusion of liposomal membrane (i.e., aggregations).

Moreover, the resultant dry powders can be stored frozen, refrigerated or even at room temperature. This is in contrast to the liquid liposomal dispersions that show significant aggregation upon accidental slow freezing. Thus, the materials described herein can be unaffected by unintentional cold chain failure. This reduces loss and facilitates longer, less expensive and less risky distribution and storage of liposomal formulations. Also, low ratios of cryoprotectant(s) to lipid were employed (i.e., down to 0.6:1 w/w, respectively). The dry powder formulations can also be administered via noninvasive routes (e.g., inhalation or intranasal) because certain thin-film freeze-dried powders with optimized compositions and processing conditions usually have good aerosol properties due to the highly porous, brittle matrix structure of the dry powders. The dry powder may also be given orally (e.g., filled into capsules).

These and other aspects of the disclosure are set out in detail below.

### I. Definitions

The abbreviations used herein have their conventional meaning within the chemical and biological arts. Description of compounds of the present disclosure is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions.

The terms “a” or “an,” as used in herein means one or more. In addition, the phrase “substituted with a[n],” as used herein, means the specified group may be substituted with one or more of any or all of the named substituents.

The terms “treating”, or “treatment” refers to any indicia of success in the treatment or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient’s physical or mental well-being. The treatment or amelioration of symptoms can be based on objective or subjective parameters, including the results of a physical examination, neuropsychiatric exams, and/or a psychiatric evaluation. For example, the certain methods presented herein successfully treat a disease associated with (e.g., caused by) an infectious agent (e.g., bacterium or virus). The term “treating” and conjugations thereof, include prevention of an injury, pathology, condition, or disease. The term “preventing” or “prevention” refers to any indicia of success in protecting a subject or patient (e.g., a subject or patient at risk of developing a disease or condition) from developing, contracting, or having a disease or condition (e.g., an infectious disease or diseases associated with an infectious agent), including preventing one or more symptoms of a disease or condition or diminishing the occurrence, severity, or duration of any symptoms of a disease or condition following administration of a prophylactic or preventative composition as described herein.

An “effective amount” is an amount sufficient for a composition (e.g., compound, vector, drug) to accomplish a stated purpose relative to the absence of the composition (e.g., compound, vector, drug) (e.g., achieve the effect for which it is administered, treat a disease (e.g., reverse or prevent or reduce severity), reduce spread of an infectious disease or agent, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a composition is an amount of a composition that, when administered to a subject, will have the intended prophylactic effect, e.g., preventing or delaying the onset (or reoccurrence) of an injury, disease (e.g., infectious disease), pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses (e.g., prime-boost). Thus, a prophylactically effective amount may be administered in one or more administrations. The exact amounts will depend on the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of infection or one or more symptoms of infection in the absence of a composition as described herein (including embodiments).

“Contacting” is used in accordance with its plain ordinary meaning and refers to the process of allowing at least two distinct species (e.g., compositions, vectors, bacterium, virus, biomolecules, or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “contacting” may include allowing two species to react, interact, or physically touch, wherein the two species may be a composition as described herein and a cell, virus, virus particle, protein, enzyme, or patient. In some embodiments contacting includes allowing a composition described herein to interact with a protein or enzyme that is involved in a signaling pathway. In some embodiments contacting includes allowing a composition described herein to interact with a component of a subject’s immune system involved in developing immunity to a component of the composition.

As defined herein, the term “inhibition”, “inhibit”, “inhibiting” and the like in reference to a protein-inhibitor or interaction means negatively affecting (e.g., decreasing) the activity or function of the protein. In some embodiments, inhibition refers to reduction of a disease or symptoms of disease. In some embodiments, inhibition refers to reduction of the growth, proliferation, or spread of an infectious agent (e.g., bacterium or virus). In some embodiments, inhibition refers to preventing the infection of a subject by an infectious agent (e.g., bacterium or virus). In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or downregulating the signaling pathway or enzymatic activity or the amount of a protein.

The term “modulator” refers to a composition that increases or decreases the level of a target (e.g., molecule, cell, bacterium, virus particle, protein) or the function of a target or the physical state of the target.

The term “modulate” is used in accordance with its plain ordinary meaning and refers to the act of changing or varying one or more properties. “Modulation” refers to the process of changing or varying one or more properties. For example, as applied to the effects of a modulator on a target, to modulate means to change by increasing or decreasing a property or function of the target or the amount of the target.

“Patient” or “subject in need thereof” refers to a living organism suffering from or prone to a disease or condition that can be treated by administration of a composition (e.g., pharmaceutical composition) as provided herein. Non-limiting examples include humans, other mammals, bovines, rats, mice, dogs, monkeys, goat, sheep, cows, deer, and other non-mammalian animals. In some embodiments, a patient is human. In some embodiments, a patient or subject in need thereof, refers to a living organism (e.g., human) at risk of developing, contracting, or having a disease or condition associated with an infectious agent (e.g., bacterium or virus).

“Disease” or “condition” refer to a state of being or health status of a patient or subject capable of being treated with the compositions or methods provided herein. In some embodiments, the disease is a disease related to (e.g., caused by) an infectious agent (e.g., bacterium or virus).

“Pharmaceutically acceptable excipient” and “pharmaceutically acceptable carrier” refer to a substance that aids the administration of an active agent to or absorption by a subject and can be included in the compositions of the present disclosure without causing a significant adverse toxicological effect on the patient. Non-limiting examples of pharmaceutically acceptable excipients include water, NaCl, normal saline solutions, lactated Ringer’s, normal sucrose, normal glucose, binders, fillers, disintegrants, lubricants, coatings, sweeteners, flavors, salt solutions (such as Ringer’s solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure. In embodiments, an excipient is a salt, buffer, detergent, polymer, amino acid, or preservative. In embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco’s modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

The term “preparation” is intended to include the formulation of the active compound with encapsulating material as a carrier providing a capsule in which the active component with or without other carriers, is surrounded by a carrier, which is thus in association with it.

As used herein, the term “administering” means oral administration, administration as a suppository, topical contact, intravenous, intraperitoneal, intramuscular, intralesional, intrathecal, intranasal, intradermal, mucosal, intrarectal, intravaginal, topical, transcutaneous, or subcutaneous administration. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example infection therapies such as antiviral drugs or antibiotics. The compositions of the disclosure can be administered alone or can be coadministered to the patient. Coadministration is meant to include simultaneous or sequential administration of the compounds individually or in combination (more than one composition). Thus, the preparations can also be combined, when desired, with other active substances. The compositions of the present disclosure can be delivered by transdermally, by a topical route, transcutaneously, formulated as solutions, suspensions, emulsions, gels, creams, ointments, pastes, jellies, paints, powders, and aerosols.

The terms “peptide,” “polypeptide,” and “protein” are used interchangeably herein to refer to a polymer of amino acid residues. The term “peptidyl” and “peptidyl moiety” means a monovalent peptide.

The term “amino acid” refers to naturally occurring and synthetic amino acids, as well as amino acid analogs. Naturally occurring amino acids are those encoded by the genetic code, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, or methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. An oligomer comprising amino acid mimetics is a peptidomimetic. A peptidomimetic moiety is a monovalent peptidomimetic.

The term “isolated” refers to a nucleic acid, polynucleotide, polypeptide, protein, or other component that is partially or completely separated from components with which it is normally associated (other proteins, nucleic acids, cells, etc.). In some embodiments, an isolated polypeptide or protein is a recombinant polypeptide or protein.

The terms “dose” and “dosage” are used interchangeably herein. A dose refers to the amount of active ingredient given to an individual at each administration. For the present methods and compositions provided herein, the dose may generally refer to the amount of disease treatment. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition and depends on the route of administration. For example, a dosage form can be in a liquid form for nebulization, e.g., for inhalants, in a tablet or liquid, e.g., for oral delivery, or a saline solution, e.g., for injection.

The terms “bind”, “bound”, “binding”, and other verb forms thereof are used in accordance with their plain ordinary meaning within Enzymology and Biochemistry and refer to the formation of one or more interactions or contacts between two compositions that may optionally interact. Binding may be intermolecular or intramolecular.

The term “associated” or “associated with” as used herein to describe a disease means that the disease is caused by, or a symptom of the disease is caused by, what is described as disease associated or what is described as associated with the disease. As used herein, what is described as being associated with a disease, if a causative agent, could be a target for treatment of the disease.

The term “portion” refers to a subset of a whole, which may also be the whole. In some embodiments, a portion is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. In some embodiments, a portion is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. Unless indicated otherwise, the term “about” in the context of a numeric value indicates the nominal value ± 10% of the nominal value. In some embodiments, “about” may be the nominal value.

### II. Compositions

In one aspect there is provided a dry powder of a liposomal formulation. In some aspects, the formulations will contain an additional agent, such as a therapeutic or diagnostic agent. The therapeutic agent may be a biological or a pharmaceutical drug. A biological may be a protein, such as an antibody, a peptide, an aptamer, an oligonucleotide, a polynucleotide, or an expression construct, such as a viral expression construct. A pharmaceutical drug may be an anti-cancer drug, such as a chemotherapeutic, an anti-viral drug, an anti-fungal drug, an antibiotic/antibacterial drug, a drug that modulates one or more aspects of cardiovascular disease, a mental health disorder, diabetes, a pulmonary disease, kidney disease, or an autoimmune disease.

In embodiments, the dry formulation includes less than 5% water. In embodiments, the dry formulation includes less than 4% water. In embodiments, the dry formulation includes less than 3% water. In embodiments, the dry formulation includes less than 2% water. In embodiments, the dry formulation includes less than 1% water. In embodiments, the dry formulation includes less than 5% water (wt/wt). In embodiments, the dry formulation includes less than 4% water (wt/wt). In embodiments, the dry formulation includes less than 3% water (wt/wt). In embodiments, the dry formulation includes less than 2% water (wt/wt). In embodiments, the dry formulation includes less than 1% water (wt/wt). In embodiments, the dry formulation includes about 5% water. In embodiments, the dry formulation includes about 4% water. In embodiments, the dry formulation includes about 3% water. In embodiments, the dry formulation includes about 2% water. In embodiments, the dry formulation includes about 1% water. In embodiments, the dry formulation includes about 5% water (wt/wt). In embodiments, the dry formulation includes about 4% water (wt/wt). In embodiments, the dry formulation includes about 3% water (wt/wt). In embodiments, the dry formulation includes about 2% water (wt/wt). In embodiments, the dry formulation includes about 1% water (wt/wt). In embodiments, the dry formulation includes less than 5% water (v/v). In embodiments, the dry formulation includes less than 4% water (v/v). In embodiments, the dry formulation includes less than 3% water (v/v). In embodiments, the dry formulation includes less than 2% water (v/v). In embodiments, the dry formulation includes less than 1% water (v/v). In embodiments, the dry formulation includes about 5% water (v/v). In embodiments, the dry formulation includes about 4% water (v/v). In embodiments, the dry formulation includes about 3% water (v/v). In embodiments, the dry formulation includes about 2% water (v/v). In embodiments, the dry formulation includes about 1% water (v/v).

In embodiments, the dry formulation includes an excipient. In embodiments, the dry formulation includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco’s modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. In embodiments, the excipient is trehalose. In embodiments, the dry formulation includes about 60% or above, wt/wt, of the excipient. In embodiments, the dry formulation includes less than 4% wt/wt of the excipient. In embodiments, the dry formulation includes less than 3% wt/wt of the excipient. In embodiments, the dry formulation includes less than 2% wt/wt of the excipient. In embodiments, the dry formulation includes less than 1% wt/wt of the excipient. In embodiments, the dry formulation includes less than 0.5% wt/wt of the excipient. In embodiments, the dry formulation includes about 5% wt/wt of the excipient. In embodiments, the dry formulation includes about 4% wt/wt of the excipient. In embodiments, the dry formulation includes about 3% wt/wt of the excipient. In embodiments, the dry formulation includes about 2% wt/wt of the excipient. In embodiments, the dry formulation includes about 1% wt/wt of the excipient. In embodiments, the dry formulation includes about 0.5% wt/wt of the excipient.

In embodiments, the dry formulation includes liposomal particles. In embodiments, the dry formulation is prepared from a liquid formulation.

In an embodiment, a powder (e.g., dry) formulation, which retains its efficacy, may be made from a liquid composition. The method includes obtaining a liquid (e.g., aqueous) composition. The liposomal composition may be frozen to obtain a frozen composition (e.g., thin film). Water is removed from the frozen composition to form a powder (e.g., dry) that includes the agent or compound.

A cryoprotectant may be added to the liposomal composition to protect the agents present in the composition (either live or dead) from damage during the freezing process. Examples of cryoprotectants include glycerol, monosaccharides, and polysaccharides (e.g., trehalose), polymers (e.g., PVP), amino acids (e.g., leucine), or proteins (e.g., human serum albumin). A cryoprotectant may be present in amounts up to about 90% by weight in the dry powder.

In another embodiment, an aqueous composition may be composed of an agent that forms particles having a particle size of less than about 200 nm (e.g., less than 10, 20, 30, 40, 50, 60, 70, 80, 90, 100, 110, 120, 130, 140, 150, 160, 170, 180, 190, 200 nm). In some embodiments, particles having a diameter of less than 200 nm (e.g., less than 10, 20, 30, 40, 50, 60, 70, 80, 90, 100, 110, 120, 130, 140, 150, 160, 170, 180, 190, 200 nm) may be used. In some embodiments, the aqueous composition can be converted to a powder, as described above, for storage, for use as an inhalant, or use in other delivery modes.

In embodiments, a dry formulation is the dry formulation described herein, including in embodiments, examples, tables, figures, and claims. In embodiments, a dry formulation is a dry formulation made by a method described herein, including in aspects, embodiments, examples, tables, figures, and claims. Provided herein is a reconstituted liquid formulation comprising a dry formulation as described herein (including in an aspect, embodiment, example, table, figure, or claim) or a dry formulation prepared using a method as described herein (including in an aspect, embodiment, example, table, figure, or claim) and a solvent (e.g., water, buffer, solution, liquid including an excipient).

Provided in another aspect is a pharmaceutical composition including a pharmaceutically acceptable excipient and any of the compositions described herein (including embodiments).

The compositions described herein (including embodiments and examples) can be administered alone or can be coadministered to the patient. Coadministration is meant to include simultaneous or sequential administration of the compositions individually or in combination (more than one composition). Thus, the preparations can also be combined, when desired, with other active substances.

Pharmaceutical compositions provided by the present disclosure include compositions wherein the active ingredient (e.g., compositions described herein, including embodiments) is contained in a therapeutically or prophylactically effective amount, i.e., in an amount effective to achieve its intended purpose. The actual amount effective for a particular application will depend, inter alia, on the condition being treated. When administered in methods to treat a disease, such compositions will contain an amount of active ingredient effective to achieve the desired result, e.g., prevent infection, and/or reducing, eliminating, or slowing the progression of disease symptoms. Determination of a therapeutically or prophylactically effective amount of a composition of the disclosure is well within the capabilities of those skilled in the art, especially in light of the detailed disclosure herein.

### III. Methods

In an aspect is provided a method for preparing a thin film including applying a liquid liposomal formulation to a freezing surface; allowing the liquid formulation to spread and freeze on the freezing surface thereby forming a thin film.

In embodiments, the liquid formulation includes an excipient. In embodiments, the liquid formulation includes a plurality of different excipients. In embodiments, the excipient is a salt, buffer, detergent, polymer, amino acid, or preservative. In embodiments, the excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco’s modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer. In embodiments, the excipient is trehalose. In embodiments, the liquid formulation includes less than 5% wt/vol of the excipient/liquid formulation. In embodiments, the liquid formulation includes less than 4% wt/vol of the excipient/liquid formulation. In embodiments, the liquid formulation includes less than 3% wt/vol of the excipient/liquid formulation. In embodiments, the liquid formulation includes less than 2% wt/vol of the excipient/liquid formulation. In embodiments, the liquid formulation includes less than 1% wt/vol of the excipient/liquid formulation. In embodiments, the liquid formulation includes less than 0.5% wt/vol of the excipient/liquid formulation. In embodiments, the liquid formulation includes about 5% wt/vol of the excipient/liquid formulation. In embodiments, the liquid formulation includes about 4% wt/vol of the excipient/liquid formulation. In embodiments, the liquid formulation includes about 3% wt/vol of the excipient/liquid formulation. In embodiments, the liquid formulation includes about 2% wt/vol of the excipient/liquid formulation. In embodiments, the liquid formulation includes about 1% wt/vol of the excipient/liquid formulation. In embodiments, the liquid formulation includes about 0.5% wt/vol of the excipient/liquid formulation. In embodiments, the liquid formulation includes about 0.1, 0.2, 0.3, 0.4, 0.5, 0.6, 0.7, 0.8, 0.9, 1, 2, 3, 4, 5, 6, 7, 8, 9, or 10% (wt/vol) of the excipient/liquid formulation. In embodiments, the liquid formulation includes 0.1, 0.2, 0.3, 0.4, 0.5, 0.6, 0.7, 0.8, 0.9, 1, 2, 3, 4, 5, 6, 7, 8, 9, or 10% (wt/vol) of the excipient/liquid formulation. In embodiments, the liquid formulation includes less than 5% of the excipient. In embodiments, the liquid formulation includes less than 4% of the excipient. In embodiments, the liquid formulation includes less than 3% of the excipient. In embodiments, the liquid formulation includes less than 2% of the excipient. In embodiments, the liquid formulation includes less than 1% of the excipient. In embodiments, the liquid formulation includes less than 0.5% of the excipient. In embodiments, the liquid formulation includes about 5% of the excipient. In embodiments, the liquid formulation includes about 4% of the excipient. In embodiments, the liquid formulation includes about 3% of the excipient. In embodiments, the liquid formulation includes about 2% of the excipient. In embodiments, the liquid formulation includes about 1% of the excipient. In embodiments, the liquid formulation includes about 0.5% of the excipient. In embodiments, the liquid formulation includes about 0.1, 0.2, 0.3, 0.4, 0.5, 0.6, 0.7, 0.8, 0.9, 1, 2, 3, 4, 5, 6, 7, 8, 9, or 10% of the excipient. In embodiments, the liquid formulation includes 0.1, 0.2, 0.3, 0.4, 0.5, 0.6, 0.7, 0.8, 0.9, 1, 2, 3, 4, 5, 6, 7, 8, 9, or 10% of the excipient.

In embodiments, the applying includes spraying or dripping droplets of the liquid formulation. In embodiments, the vapor-liquid interface of the droplets is less than 500 cm⁻¹ area/volume. In embodiments, the vapor-liquid interface of the droplets is less than 400 cm⁻¹ area/volume. In embodiments, the vapor-liquid interface of the droplets is less than 300 cm⁻¹ area/volume. In embodiments, the vapor-liquid interface of the droplets is less than 200 cm⁻¹ area/volume. In embodiments, the vapor-liquid interface of the droplets is less than 100 cm⁻¹ area/volume. In embodiments, the vapor-liquid interface of the droplets is less than 50 cm⁻¹ area/volume. In embodiments, the vapor-liquid interface of the droplets is less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹ area/volume.

In embodiments, the method further includes contacting the droplets with a freezing surface having a temperature below the freezing temperature of the liquid formulation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C. below the freezing temperature). In embodiments, the method further includes contacting the droplets with a freezing surface having a temperature differential of at least 30° C. between the droplets and the surface. In embodiments, the temperature differential is at least 40° C. between the droplets and the surface. In embodiments, the temperature differential is at least 50° C. between the droplets and the surface. In embodiments, the temperature differential is at least 60° C. between the droplets and the surface. In embodiments, the temperature differential is at least 70° C. between the droplets and the surface. In embodiments, the temperature differential is at least 80° C. between the droplets and the surface. In embodiments, the temperature differential is at least 90° C. between the droplets and the surface. In embodiments, the temperature differential between the droplets and the surface is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 180 or 200° C.

In embodiments, the thin film has a thickness of about 5 mm, about 4 mm, about 3 mm, about 2 mm, about 1 mm, less than 3 mm, less than 2 mm, less than 1 mm, less than 500 micrometers. In embodiments, the thin film has a thickness of less than 400 micrometers. In embodiments, the thin film has a thickness of less than 300 micrometers. In embodiments, the thin film has a thickness of less than 200 micrometers. In embodiments, the thin film has a thickness of less than 100 micrometers. In embodiments, the thin film has a thickness of less than 50 micrometers. In embodiments, the thin film has a thickness of less than 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers. In embodiments, the thin film has a thickness of about 500 micrometers. In embodiments, the thin film has a thickness of about 400 micrometers. In embodiments, the thin film has a thickness of about 300 micrometers. In embodiments, the thin film has a thickness of about 200 micrometers. In embodiments, the thin film has a thickness of about 100 micrometers. In embodiments, the thin film has a thickness of about 50 micrometers. In embodiments, the thin film has a thickness of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 micrometers.

In embodiments, the thin film has a surface area to volume ratio of between about 5 and 500 cm⁻¹. In embodiments, the thin film has a surface area to volume ratio of between 25 and 400 cm⁻¹. In embodiments, the thin film has a surface area to volume ratio of between 25 and 300 cm⁻ ¹. In embodiments, the thin film has a surface area to volume ratio of between 25 and 200 cm⁻¹. In embodiments, the thin film has a surface area to volume ratio of between 25 and 100 cm⁻¹. In embodiments, the thin film has a surface area to volume ratio of between 100 and 500 cm⁻¹. In embodiments, the thin film has a surface area to volume ratio of between 200 and 500 cm⁻¹. In embodiments, the thin film has a surface area to volume ratio of between 300 and 500 cm⁻¹. In embodiments, the thin film has a surface area to volume ratio of between 400 and 500 cm⁻¹. In embodiments, the thin film has a surface area to volume ratio of between 100 and 400 cm⁻¹. In embodiments, the thin film has a surface area to volume ratio of between 200 and 300 cm⁻¹. In embodiments, the thin film has a surface area to volume ratio of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 cm⁻¹. In embodiments, the thin film has a surface area to volume ratio of between about 25 and about 500 cm⁻¹. In embodiments, the thin film has a surface area to volume ratio of between about 25 and about 400 cm⁻¹. In embodiments, the thin film has a surface area to volume ratio of between about 25 and about 300 cm⁻¹. In embodiments, the thin film has a surface area to volume ratio of between about 25 and about 200 cm⁻¹. In embodiments, the thin film has a surface area to volume ratio of between about 25 and about 100 cm⁻¹. In embodiments, the thin film has a surface area to volume ratio of between about 100 and about 500 cm⁻¹. In embodiments, the thin film has a surface area to volume ratio of between about 200 and about 500 cm⁻¹. In embodiments, the thin film has a surface area to volume ratio of between about 300 and about 500 cm⁻¹. In embodiments, the thin film has a surface area to volume ratio of between about 400 and about 500 cm⁻¹. In embodiments, the thin film has a surface area to volume ratio of between about 100 and about 400 cm⁻¹. In embodiments, the thin film has a surface area to volume ratio of between about 200 and about 300 cm⁻¹.

In embodiments, the freezing rate of the droplets is between about 10 K/second and about 10⁵ K/second. In embodiments, the freezing rate of the droplets is between about 10 K/second and about 10⁴ K/second. In embodiments, the freezing rate of the droplets is between about 10 K/second and about 10³ K/second. In embodiments, the freezing rate of the droplets is between about 10² K/second and about 10³ K/second. In embodiments, the freezing rate of the droplets is between about 50 K/second and about 5×10² K/second. In embodiments, the freezing rate of the droplets is between 10 K/second and10⁵ K/second. In embodiments, the freezing rate of the droplets is between 10 K/second and10⁴ K/second. In embodiments, the freezing rate of the droplets is between 10 K/second and10³ K/second. In embodiments, the freezing rate of the droplets is between 10² K/second and10³ K/second. In embodiments, the freezing rate of the droplets is between 50 K/second and 5×10² K/second. In embodiments, the freezing rate of the droplets is about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, the freezing rate of the droplets is 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 K/second. In embodiments, each of the droplets freezes upon contact with the freezing surface in less than about 50, 75, 100, 125, 150, 175, 200, 250, 500, 1,000, or 2,000 milliseconds. In embodiments, each of the droplets freezes upon contact with the freezing surface in less than 50, 75, 100, 125, 150, 175, 200, 250, 500, 1,000, or 2,000 milliseconds.

In embodiments, the droplets have an average diameter between about 0.1 and about 5 mm, between about 2 and about 25° C. In embodiments, the droplets have an average diameter between about 2 and about 4 mm, between about 20 and about 25° C. In embodiments, the droplets have an average diameter between about 1 and about 4 mm, between about 2 and about 25° C. In embodiments, the droplets have an average diameter between about 2 and about 3 mm, between about 2 and about 25° C. In embodiments, the droplets have an average diameter between about 1 and about 3 mm, between about 2 and about 25° C. In embodiments, the droplets have an average diameter between about 1 and about 2 mm, between about 2 and about 25° C. In embodiments, the droplets have an average diameter between about 3 and about 4 mm, between about 2 and about 25° C. In embodiments, the droplets have an average diameter between 0.1 and 5 mm, between 2 and 25° C. In embodiments, the droplets have an average diameter between 2 and 4 mm, between 2 and 25° C. In embodiments, the droplets have an average diameter between 1 and 4 mm, between 2 and 25° C. In embodiments, the droplets have an average diameter between 2 and 3 mm, between 2 and 25° C. In embodiments, the droplets have an average diameter between 1 and 3 mm, between 2 and 25° C. In embodiments, the droplets have an average diameter between 1 and 2 mm, between 2 and 25° C. In embodiments, the droplets have an average diameter between 3 and 4 mm, between 2° and 25° C.

In embodiments, the method further includes removing the solvent (e.g., water or liquid) from the thin film to form a dry liposomal formulation.

In embodiments, is a method of making a dry formulation from a thin film (e.g., including a thin film made using a method as described herein), including removing the solvent (e.g., water or liquid) from the thin film to form a dry formulation. In embodiments of the methods described herein, the dry formulation is a dry formulation as described herein, including in an aspect, embodiment, example, table, figure, or claim. In embodiments, a method of making a thin film or a method of making dry formulation is used to make a dry formulation as described herein, including in an aspect, embodiment, example, table, figure, or claim.

In embodiments, the removing of the solvent includes lyophilization. In embodiments, the removing of the solvent includes lyophilization at temperatures of -20° C. or less. In embodiments, the removing of the solvent includes lyophilization at temperatures of -25° C. or less. In embodiments, the solvent includes lyophilization at temperatures of -40° C. or less. In embodiments, the removing of the solvent includes lyophilization at temperatures of -50° C. or less. In embodiments, the removing of the solvent includes lyophilization at temperatures of about -20° C. or less. In embodiments, the removing of the solvent includes lyophilization at temperatures of about -25° C. or less. In embodiments, the solvent includes lyophilization at temperatures of about -40° C. or less. In embodiments, the removing of the solvent includes lyophilization at temperatures of about -50° C. or less. Primary drying can be performed at -20° C. to -50° C., and secondary drying can be performed at 4-25° C.

In embodiments, the method further includes solvating, reconstituting or rehydrating the dry formulation thereby forming a reconstituted liquid formulation. A reconstituted liquid formulation may also be called a solvated dry formulation.

In embodiments, is a method of making a reconstituted liquid formulation from a dry formulation (e.g., including a dry formulation made using a method as described herein), including solvating a dry formulation and thereby forming a reconstituted liquid formulation. In embodiments of the methods described herein, the dry formulation is a dry formulation as described herein, including in an aspect, embodiment, example, table, figure, or claim. In embodiments, a method of making a thin film, a method of making a dry formulation, or a method of reconstituting a liquid formulation is used to make a reconstituted liquid formulation as described herein, including in an aspect, embodiment, example, table, figure, or claim.

In embodiments, the reconstituted liquid formulation includes particles. In embodiments, the particles have an average diameter of between about 10 nm and about 2 µm. In embodiments, the particles have an average diameter of between about 20 nm and about 2 µm. In embodiments, the particles have an average diameter of between about 50 nm and about 2 µm. In embodiments, the particles have an average diameter of between about 100 nm and about 2 µm. In embodiments, the particles have an average diameter of between about 200 nm and about 2 µm. In embodiments, the particles have an average diameter of between about 500 nm and about 2 µm. In embodiments, the particles have an average diameter of between about 1 µm and about 2 µm. In embodiments, the particles have an average diameter of between about 10 nm and about 1 µm. In embodiments, the particles have an average diameter of between about 10 nm and about 500 nm. In embodiments, the particles have an average diameter of between about 10 nm and about 200 nm. In embodiments, the particles have an average diameter of between about 10 nm and about 200 nm. In embodiments, the particles have an average diameter of between about 10 nm and about 100 nm. In embodiments, the particles have an average diameter of between about 10 nm and about 50 nm. In embodiments, the particles have an average diameter of between about 10 nm and about 20 nm. In embodiments, the particles have an average diameter of between about 20 nm and about 1 µm. In embodiments, the particles have an average diameter of between about 50 nm and about 500 nm. In embodiments, the particles have an average diameter of between about 100 nm and about 500 nm. In embodiments, the particles have an average diameter of between about 100 nm and about 200 nm. In embodiments, the reconstituted liquid formulation includes particles. In embodiments, the particles have an average diameter of between 10 nm and 2 µm. In embodiments, the particles have an average diameter of between 20 nm and 2 µm. In embodiments, the particles have an average diameter of between 50 nm and 2 µm. In embodiments, the particles have an average diameter of between 100 nm and 2 µm. In embodiments, the particles have an average diameter of between 200 nm and 2 µm. In embodiments, the particles have an average diameter of between 500 nm and 2 µm. In embodiments, the particles have an average diameter of between 1 µm and 2 µm. In embodiments, the particles have an average diameter of between 10 nm and 1 µm. In embodiments, the particles have an average diameter of between 10 nm and 500 nm. In embodiments, the particles have an average diameter of between 10 nm and 200 nm. In embodiments, the particles have an average diameter of between 10 nm and 200 nm. In embodiments, the particles have an average diameter of between 10 nm and 100 nm. In embodiments, the particles have an average diameter of between 10 nm and 50 nm. In embodiments, the particles have an average diameter of between 10 nm and 20 nm. In embodiments, the particles have an average diameter of between 20 nm and 1 µm. In embodiments, the particles have an average diameter of between 50 nm and 500 nm. In embodiments, the particles have an average diameter of between 100 nm and 500 nm. In embodiments, the particles have an average diameter of between 100 nm and 200 nm.

In embodiments, the reconstituted liquid formulation includes particles of the same average diameter as the liquid formulation (prior to forming the dry formulation from the liquid formulation) particles. In embodiments, the reconstituted liquid formulation includes particles having an average diameter within 5% of the average diameter of particles in the liquid formulation (prior to forming the dry formulation from the liquid formulation). In embodiments, the reconstituted liquid formulation includes particles having an average diameter within 10% of the average diameter of particles in the liquid formulation (prior to forming the dry formulation from the liquid formulation). In embodiments, the reconstituted liquid formulation includes particles having an average diameter within 20% of the average diameter of particles in the liquid formulation (prior to forming the dry formulation from the liquid formulation). In embodiments, the reconstituted liquid formulation includes particles having an average diameter within 10%, 20%, 30% or 40% of the average diameter of particles in the liquid formulation (prior to forming the dry formulation from the liquid formulation). There may less than 5% or 10% of the particles aggregated (e.g., 8 particles can aggregate together to form a single particle with a diameter approximately twice that of the original smaller particles). In embodiments, the reconstituted liquid formulation includes particles having an average diameter within about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% of the average diameter of particles in the liquid formulation (prior to forming the dry formulation from the liquid formulation). In embodiments, the reconstituted liquid formulation includes particles having an average diameter within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30% of the average diameter of particles in the liquid formulation (prior to forming the dry formulation from the liquid formulation).

In embodiments, the solvating, reconstituting or rehydrating of the dry formulation is at least one day after preparing the dry formulation from the liquid formulation (e.g., the dry formulation is stored for at least one day). In embodiments, the solvating of the dry formulation is at least two days after preparing the dry formulation from the liquid formulation (e.g., the dry formulation is stored for at least two days). In embodiments, the solvating of the dry formulation is at least three days after preparing the dry formulation from the liquid formulation (e.g., the dry formulation is stored for at least three days). In embodiments, the solvating of the dry formulation is at least one week after preparing the dry formulation from the liquid formulation (e.g., the dry formulation is stored for at least one week). In embodiments, the solvating of the dry formulation is at least two weeks after preparing the dry formulation from the liquid formulation (e.g., the dry formulation is stored for at least two weeks). In embodiments, the solvating of the dry formulation is at least one month after preparing the dry formulation from the liquid formulation (e.g., the dry formulation is stored for at least one month). In embodiments, the solvating of the dry formulation is at least two months after preparing the dry formulation from the liquid formulation (e.g., the dry formulation is stored for at least two months). In embodiments, the solvating of the dry formulation is at least three months after preparing the dry formulation from the liquid formulation (e.g., the dry formulation is stored for at least three months). In embodiments, the solvating of the dry formulation is at least six months after preparing the dry formulation from the liquid formulation (e.g., the dry formulation is stored for at least six months). In embodiments, the solvating of the dry formulation is at least one year after preparing the dry formulation from the liquid formulation (e.g., the dry formulation is stored for at least one year). In embodiments, the solvating of the dry formulation is at least two years after preparing the dry formulation from the liquid formulation (e.g., the dry formulation is stored for at least two years). In embodiments, the solvating of the dry formulation is at least three years after preparing the dry formulation from the liquid formulation (e.g., the dry formulation is stored for at least three years). In embodiments, the solvating of the dry formulation is at least five years after preparing the dry formulation from the liquid formulation (e.g., the dry formulation is stored for at least five years). In embodiments, the solvating of the dry formulation is at least ten years after preparing the dry formulation from the liquid formulation (e.g., the dry formulation is stored for at least ten years).

In embodiments, prior to the solvating, reconstituting or rehydrating of the dry formulation, the dry formulation is stored at about 4° C. for at least 99% of the time. In embodiments, prior to the solvating of the dry formulation, the dry formulation is stored at less than 4° C. for at least 99% of the time. In embodiments, prior to the solvating of the dry formulation, the dry formulation is stored at less than 0° C. for at least 99% of the time. In embodiments, prior to the solvating of the dry formulation, the dry formulation is stored at less than -20° C. for at least 99% of the time. In embodiments, prior to the solvating of the dry formulation, the dry formulation is stored at about -20° C. for at least 99% of the time. In embodiments, prior to the solvating of the dry formulation, the dry formulation is stored at less than -80° C. for at least 99% of the time. In embodiments, prior to the solvating of the dry formulation, the dry formulation is stored at about -80° C. for at least 99% of the time. In embodiments, prior to the solvating of the dry formulation, the dry formulation is stored at ambient temperatures (e.g., room temperature). In embodiments, prior to the solvating of the dry formulation, the dry formulation is stored at between 20 and 24° C. for at least 99% of the time. In embodiments, prior to the solvating of the dry formulation, the dry formulation is stored at between 4 and 24° C. for at least 99% of the time. In embodiments, prior to the solvating of the dry formulation, the dry formulation is stored at between 0 and 24° C. for at least 99% of the time. In embodiments, prior to the solvating of the dry formulation, the dry formulation is stored at between 4 and 40° C. for at least 99% of the time. In embodiments, prior to the solvating of the dry formulation, the dry formulation is stored at between 0 and 40° C. for at least 99% of the time. In embodiments, prior to the solvating of the dry formulation, the dry formulation is stored at about 4° C. for at least 90% of the time. In embodiments, prior to the solvating of the dry formulation, the dry formulation is stored at less than 4° C. for at least 90% of the time. In embodiments, prior to the solvating of the dry formulation, the dry formulation is stored at less than 0° C. for at least 90% of the time. In embodiments, prior to the solvating of the dry formulation, the dry formulation is stored at less than -20° C. for at least 90% of the time. In embodiments, prior to the solvating of the dry formulation, the dry formulation is stored at between 20 and 24° C. for at least 90% of the time. In embodiments, prior to the solvating of the dry formulation, the dry formulation is stored at between 4 and 24° C. for at least 90% of the time. In embodiments, prior to the solvating of the dry formulation, the dry formulation is stored at between 0 and 24° C. for at least 90% of the time. In embodiments, prior to the solvating of the dry formulation, the dry formulation is stored at between 4 and 40° C. for at least 90% of the time. In embodiments, prior to the solvating of the dry formulation, the dry formulation is stored at between 0 and 40° C. for at least 90% of the time.

In embodiments, upon solvating, reconstituting or rehydrating the dry formulation the resulting reconstituted liquid formulation remains homogeneous. As used in reference to the status of a reconstituted liquid formulation, the term “homogenous” refers to a lack of a significant amount of aggregation and/or precipitation forming, such that the reconstituted liquid formulation does not include solid matter that is not evenly dispersed (e.g., solid matter visible to the naked eye, solid matter that settles in the liquid, solid matter that was not apparent in a liquid formulation prior to formation of the dry formulation and reconstitution, precipitate that was not present in the liquid formulation prior to formation of the dry formulation). In embodiments, upon solvating the dry formulation the resulting reconstituted liquid formulation remains homogeneous for at least one day. In embodiments, upon solvating the dry formulation the resulting reconstituted liquid formulation remains homogeneous for at least two days. In embodiments, upon solvating the dry formulation the resulting reconstituted liquid formulation remains homogeneous for at least three days. In embodiments, upon solvating the dry formulation the resulting reconstituted liquid formulation remains homogeneous for at least one week. In embodiments, upon solvating the dry formulation the resulting reconstituted liquid formulation remains homogeneous for at least two weeks. In embodiments, upon solvating the dry formulation the resulting reconstituted liquid formulation remains homogeneous for at least one month. In embodiments, upon solvating the dry formulation the resulting reconstituted liquid formulation remains homogeneous for at least three months. In embodiments, upon solvating the dry formulation the resulting reconstituted liquid formulation remains homogeneous for at least six months. In embodiments, upon solvating the dry formulation the resulting reconstituted liquid formulation remains homogeneous for at least one year. In embodiments, upon solvating the dry formulation the resulting reconstituted liquid formulation does not form a precipitate (e.g., solid matter visible to the naked eye, solid matter that settles in the liquid, solid matter that was not apparent in a liquid formulation prior to formation of the dry formulation and reconstitution, precipitate that was not present in the liquid formulation prior to formation of the dry formulation). In embodiments, upon solvating the dry formulation the resulting reconstituted liquid formulation does not form a precipitate for at least one day. In embodiments, upon solvating the dry formulation the resulting reconstituted liquid formulation does not form a precipitate for at least two days. In embodiments, upon solvating the dry formulation the resulting reconstituted liquid formulation does not form a precipitate for at least three days. In embodiments, upon solvating the dry formulation the resulting reconstituted liquid formulation does not form a precipitate for at least one week. In embodiments, upon solvating the dry formulation the resulting reconstituted liquid formulation does not form a precipitate for at least two weeks. In embodiments, upon solvating the dry formulation the resulting reconstituted liquid formulation does not form a precipitate for at least one month. In embodiments, upon solvating the dry formulation the resulting reconstituted liquid formulation does not form a precipitate for at least three months. In embodiments, upon solvating the dry formulation the resulting reconstituted liquid formulation does not form a precipitate for at least six months. In embodiments, upon solvating the dry formulation the resulting reconstituted liquid formulation does not form a precipitate for at least one year.

In embodiments, the precipitate includes particles having an average diameter greater than 50 µm. In embodiments, the precipitate includes particles having an average diameter greater than 100 µm. In embodiments, the precipitate includes particles having an average diameter greater than 200 µm. In embodiments, the precipitate includes particles having an average diameter greater than 300 µm. In embodiments, the precipitate includes particles having an average diameter greater than 400 µm. In embodiments, the precipitate includes particles having an average diameter greater than 500 µm. In embodiments, the precipitate includes particles having an average diameter greater than 600 µm. In embodiments, the precipitate includes particles having an average diameter greater than 700 µm. In embodiments, the precipitate includes particles having an average diameter greater than 800 µm. In embodiments, the precipitate includes particles having an average diameter greater than 900 µm. In embodiments, the precipitate includes particles having an average diameter greater than 1000 µm. In embodiments, the precipitate includes particles having an average diameter greater than about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 µm. In embodiments, the precipitate includes particles having an average diameter of about 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 µm. In embodiments, the precipitate includes particles having an average diameter greater than 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 µm. In embodiments, the precipitate (that is not formed) includes particles having an average diameter of 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 µm.

In an aspect is provided a method of treating a disease in a patient in need of such treatment, the method including administering a therapeutically effective amount of a solvated dry formulation as described herein to the patient.

In an aspect is provided a method of treating a disease in a patient in need of such treatment, the method including administering a therapeutically effective amount of dry formulations described herein (e.g., in an aspect, embodiment, example, table, figure, or claims).

In embodiments, the dry formulation is administered by inhalation, intradermally, orally, or vaginally. In embodiments, the dry formulation is administered through the nasal mucosa, bronchoalveolar mucosa, or gastrointestinal mucosa.

In embodiments, the method is a method described herein, including in an aspect, embodiment, example, table, figure, or claim. Provided herein is a method of preparing a dry formulation including a method of preparing a thin film as described herein (including in an aspect, embodiment, example, table, figure, or claim) and a method of removing a solvent from a thin film as described herein (including in an aspect, embodiment, example, table, figure, or claim). Provided herein is a method of preparing a reconstituted dry formulation including a method of preparing a dry formulation as described herein (including in an aspect, embodiment, example, table, figure, or claim), a method of preparing a thin film as described herein (including in an aspect, embodiment, example, table, figure, or claim) and a method of removing a solvent from a thin film as described herein (including in an aspect, embodiment, example, table, figure, or claim).

In embodiments, to form a powder, an aqueous liposomal composition is first frozen to form a frozen composition, then the frozen water is removed to form the powder. A fast-freezing process is used to form the frozen composition. A fast-freezing process, as used herein, is a process that can freeze a thin film of liquid (less than about 500 microns or 2-4 mm) in a time of less than or equal to about 3000 milliseconds. In the TFF process liquid droplets fall from a given height and impact, spread, and freeze on a cooled solid substrate. Typically, the substrate is a metal drum that is cooled to below 250 K, or below 200 K or below 150 K. On impact the droplets that are deformed into thin films freeze in a time of between about 70 ms and 3000 ms. The frozen thin films may be removed from the substrate by a stainless-steel blade mounted along the rotating drum surface. The frozen thin films are collected in liquid nitrogen to maintain in the frozen state. Further details regarding thin film freezing processes may be found in the paper to Engstrom et al. “Formation of Stable Submicron Protein Particles by Thin Film Freezing” Pharmaceutical Research, Vol. 25, No. 6, June 2008, 1334-1346, which is incorporated herein by reference.

Water (e.g., frozen water) is removed from the frozen composition to produce a dry powder. Water (e.g., frozen water) may be removed by a lyophilization process or a freeze-drying process. Water may also be removed by an atmospheric freeze-drying process.

The resulting powder can be readily reconstituted to form a stable dispersion without significant loss of stability or activity. The powder may be transported and stored in a wide range of temperatures without concern of accidental exposure to freezing conditions. In addition, the powder may also be stored at room temperature, which will potentially decrease the costs of liposomal materials. In fact, it is generally less costly to transport dry solid powder than liquid.

Described herein are compositions and methods for preparing a thin film or a dry formulation by spraying or dripping droplets of a liquid formulation such that the formulation is exposed to an vapor-liquid interface of less than 500 cm⁻¹ area/volume, such as 25 to 500 cm⁻¹ (e.g., less than 50, 100, 150, 200, 250, 300, 400) and contacting the droplet with a freezing surface having a temperature lower than the freezing temperature of the liquid formulation (e.g., has a temperature differential of at least 30° C. between the droplet and the surface), wherein the surface freezes the droplet into a thin film with a thickness of less than 5 mm, such as about 2-4 mm, about 1 mm, about 500 micrometers (e.g., 450, 400, 350, 300, 250, 200, 150, 100, or 50 micrometers). In embodiments, the method may further include the step of removing the liquid (e.g., solvent, water) from the frozen material to form a dry formulation (e.g., particles). In embodiments, the droplets freeze upon contact with the surface in less than 50, 75, 100, 125, 150, 175, 200, 250, 500, 1,000, 2,000, or 3000 milliseconds. In embodiments, the droplets freeze upon contact with the surface in less than 50 or 150 milliseconds. In embodiments, the droplet has a diameter between 2 and 5 mm at room temperature. In embodiments, the droplet forms a thin film on the freezing surface of between 50 micrometers and 5 mm, such as 2-4 mm in thickness. In embodiments, the droplets have a cooling rate of between 50-250 K/s. In embodiments, the particles of the dry formulation, after liquid (e.g., solvent or water) removal, have a surface area of at least 10, 15, 25, 50, 75, 100, 125, 150 or 200 m²/gr (e.g., surface area of 10, 15, 25, 50, 75, 100, 125, 150 or 200 m²/gr). Minimizing gas-liquid interface can improve protein stability by limiting the amount of protein that can adsorb to the interface.

In embodiments, the droplets may be delivered to the cold or freezing surface in a variety of manners and configurations. In embodiments, the droplets may be delivered in parallel, in series, at the center, middle or periphery or a platen, platter, plate, roller, conveyor surface. In embodiments, the freezing or cold surface may be a roller, a belt, a solid surface, circular, cylindrical, conical, oval and the like that permit for the droplet to freeze. For a continuous process a belt, platen, plate or roller may be particularly useful. In embodiments, the frozen droplets may form beads, strings, films or lines of frozen liquid formulation. In embodiments, the effective ingredient is removed from the surface with a scraper, wire, ultrasound or other mechanical separator prior to the lyophilization process. Once the material is removed from the surface of the belt, platen, roller or plate the surface is free to receive additional material.

In embodiments, the surface is cooled by a cryogenic solid, a cryogenic gas, a cryogenic liquid or a heat transfer fluid capable of reaching cryogenic temperatures or temperatures below the freezing point of the liquid formulation (e.g., at least 30° C. less than the temperature of the droplet). In embodiments, the liquid formulation further includes one or more excipients selected from surfactants, polymeric surfactants, vesicles, polymers, including copolymers and homopolymers and biopolymers, dispersion aids, and serum albumin. In embodiments, the temperature differential between the droplet and the surface is at least 30° C. In embodiments, the excipients or stabilizers that can be included in the liquid formulations that are to be frozen as described herein include: cryoprotectants, lyoprotectants, surfactants, fillers, stabilizers, polymers, protease inhibitors, antioxidants and absorption enhancers. Specific nonlimiting examples of excipients that may be included in the formulations described herein include: sucrose, trehalose, Span 80, Tween 80, Brij 35, Brij 98, Pluronic, sucroester 7, sucroester 11, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, glycolic acid and salts, deoxycholic acid and salts, sodium fusidate, cyclodextrins, polyethylene glycols, labrasol, polyvinyl alcohols, polyvinyl pyrrolidones and tyloxapol.

In embodiments, the method may further include the step of removing the liquid (e.g., solvent or water) from the frozen liquid formulation to form a dry formulation. In embodiments, the solvent further includes at least one or more excipient or stabilizers selected from, e.g., surfactants, polymeric surfactants, vesicles, polymers, including copolymers and homopolymers and biopolymers, dispersion aids, and serum albumin. In embodiments, the temperature differential between the solvent and the surface is at least about 30° C.

In embodiments, the resulting powder can be redispersed into a suitable aqueous medium such as saline, buffered saline, water, buffered aqueous media, solutions of amino acids, solutions of vitamins, solutions of carbohydrates, or the like, as well as combinations of any two or more thereof, to obtain a suspension that can be administered to mammals (e.g., humans).

In embodiments, is described a single-step, single-vial method for preparing a thin film or dry formulation by reducing the temperature of a vial wherein the vial has a temperature below the freezing temperature of a liquid formulation (e.g., a temperature differential of at least 30° C. between the liquid formulation and the vial) and spraying or dripping droplets of a liquid formulation directly into the vial such that the liquid formulation is exposed to a vapor-liquid interface of less than 500 cm⁻¹ area/volume, wherein the surface freezes the droplet into a thin film with a thickness of less than 500 micrometers and a surface area to volume between 25 to 500 cm⁻ ¹. In embodiments, the droplets freeze upon contact with the surface in less than about 50, 75, 100, 125, 150, 175, 200, 250, 500, 1,000, 2,000 or 3000 milliseconds (e.g., in about 50, 75, 100, 125, 150, 175, 200, 250, 500, 1,000 or 2,000 or 3000 milliseconds), and may freeze upon contact with the surface in about 50 or 150 to 500 milliseconds. In embodiments, a droplet has a diameter between 0.1 and 5 mm at room temperature (e.g., a diameter between 2 and 4 mm at room temperature). In embodiments, the droplet forms a thin film on the surface of between 50 micrometers to 5 mm, such as about 2-4 millimeters in thickness. In embodiments, the droplets have a cooling rate of between 50-250 K/s. In embodiments, the vial may be cooled by a cryogenic solid, a cryogenic gas, a cryogenic liquid, a freezing fluid, a freezing gas, a freezing solid, a heat exchanger, or a heat transfer fluid capable of reaching cryogenic temperatures or temperatures below the freezing point of the liquid formulation. In embodiments, the vial may be rotated as the spraying or droplets are delivered to permit the layering or one or more layers of the liquid formulation. In embodiments, the vial and the liquid formulation are pre-sterilized prior to spraying or dripping. In embodiments, the step of spraying or dripping is repeated to overlay one or more thin films on top of each other to fill the vial to any desired level up to totally full.

### IV. Examples

The following examples as well as the figures are included to demonstrate preferred embodiments of the disclosure. It should be appreciated by those of skill in the art that the techniques disclosed in the examples or figures represent techniques discovered by the inventors to function well in the practice of the disclosure and thus can be considered to constitute preferred modes for its practice. However, those of skill in the art should, in light of the disclosure, appreciate that many changes can be made in the specific embodiments which are disclosed and still obtain a like or similar result without departing from the spirit and scope of the disclosure.

### Example 1: Dry Powder Compositions of Neutral, Anionic, and Cationic Liposomes

Materials and Methods: Liposomes were prepared by thin film hydration method as described previously with some modifications (Yanasarn et al., 2011). To prepare neutral liposomes, a thin layer of 1,2-dioleoyl-sn-glycero-3-phosphocholine (DOPC, Avanti, Alabaster, AL, US) and cholesterol (1:1 molar ratio, 5 mg total lipids) was formed on the wall of a 7-mL glass vial by chloroform evaporation under gentle stream of nitrogen gas. The lipid thin film was hydrated in 1 mL of milli-Q water and the vials were vortex-mixed for 30 s. Then, the vials were stirred overnight at 1200 rpm using a magnetic stirrer followed by sonication for 5 min. For the preparation of anionic and cationic liposomes, DOPC was replaced with either 1,2-dioleoyl-sn-glycero-3-phosphate sodium salt (DOPA) or 1,2-Dioleoyl-3-trimethylammonium-propane chloride salt (DOTAP), respectively.

Liposome dispersions were converted to dry powders by thin-film freeze-drying (TFFD) technology using different cryoprotectants including sucrose, trehalose, and mannitol at lipid to cryoprotectant ratio of 1:8, 1:15 or 1:30 w/w, respectively. Briefly, the liposome and cryoprotectant dispersion (125 µL) was dropped onto a cryogenically cooled surface to form frozen thin films rapidly. The frozen films were then lyophilized using a VirTis Advantage bench top tray lyophilizer (The VirTis Company, Inc. Gardiner, NY). Lyophilization was performed over 60 h at pressures ≤ 100 mTorr. The shelf temperature was maintained at -40° C. for 20 h then gradually ramped to +25° C., over 20 h. Throughout the secondary drying phase, the vials were kept at +25° C. for additional 20 h. The vials were then stored at room temperature for future use.

Dynamic light scattering (DLS) using a Malvern Zeta Sizer Nano ZS (MA, US) was employed to determine the particle size distribution of liquid liposome dispersions (i.e., before TFFD) and rehydrated dry powders prepared by TFFD.

Results: Firstly, the hypothesis that the rapid freezing rate of TFFD process can protect the liposomes against slow freezing-induced membrane rupture was validated using neutral (i.e., DOPC-Cholesterol) liposomes. Sucrose was employed as a cryoprotectant at total lipids to sucrose ratio of 1:16 w/w. As, shown in FIG. 1, the liposome mean particle size and particle size distribution was maintained after TFFD. The rapid freezing rate of TFFD process may have resulted in the formation of small ice crystals and uniform distribution of the cytoprotectant which in turn diminish various freezing induced stresses (e.g., liposome concentration and phase separation).

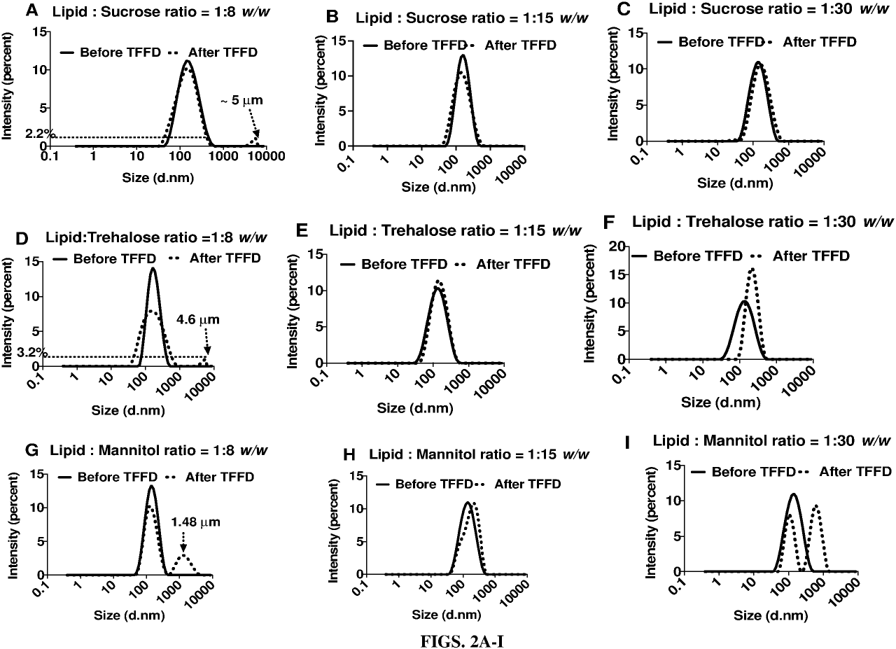

Then, the hypothesis was further validated using anionic and cationic liposomes and different cryoprotectants at different cryoprotectant to total lipid ratios. Relatively low cryoprotectant to total lipid ratio (i.e., 8:1 w/w, respectively) failed to protect the anionic liposome membrane against freezing and/or lyophilization-induced stresses. As depicted in FIGS. 2A, 2D and 2G, liposome fusion was induced may be due to liposome concentration or phase separation during freezing. FIG. 2G also shows a higher extent of liposome fusion when mannitol was employed as a cytoprotectant compared to either sucrose or trehalose at the same cryoprotectant to total lipid ratio (i.e., 8:1 w/w, respectively). When the cryoprotectant to lipid ratio was increased to 15:1 w/w, respectively, sucrose, trehalose, and mannitol successfully protected the anionic liposomes against various stresses encountered during the freezing and/or the drying steps and thus maintained the anionic liposome particle size distribution after TFFD and reconstitution. At a cryoprotectant to total lipid ratio of 30:1 w/w, respectively, only sucrose maintained the anionic liposome particle size distribution after TFFD and reconstitution. Trehalose and mannitol showed either significant increase of the liposomes mean particle size or aggregation, respectively (FIGS. 2F and 2I).

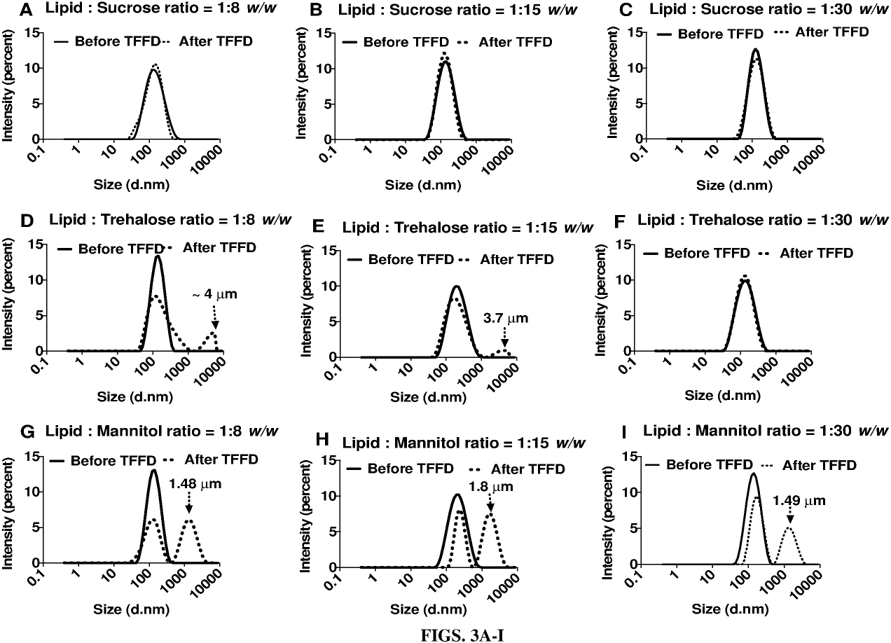

The applicability of TFFD process for converting cationic liposomes to dry powders while maintaining their mean particle size after reconstitution was also investigated. As depicted in FIGS. 3A-C, sucrose at all screened ratios (i.e., sucrose to total lipid ratios of 1:8 w/w, 1:15 w/w and 1:30 w/w) was effective in protecting the cationic liposomes against various freezing and/or drying-induced stresses and thus the cationic liposome mean particle size was maintained after TFFD and reconstitution. Trehalose was effective in maintaining the cationic liposome mean particle size at trehalose to total lipid ratio of 30:1 w/w, respectively. At lower weight ratios, a concentration dependent liposome aggregation was observed (FIGS. 3D-E). On the other hand, mannitol was ineffective in maintaining the cationic liposome mean particle size after TFFD at all the screened ratios (FIGS. 3G-I).

Overall, neutral, anionic and cationic liposomes were shown to be processed into dry powders with enhanced stability profile compared to liquid liposomal dispersions while maintaining their mean particle size after reconstitution. The effectiveness of TFFD process in maintaining the liposomes’ mean particle size after reconstitution depends on the physicochemical characteristics of the employed cryoprotectant and lipids as well as the total lipids to cryoprotectant weight/molar ratios. Sucrose was the most effective cryoprotectant in maintaining the liposomes’ mean particle size after TFFD and reconstitution followed by trehalose. While the utility of mannitol varies depending on the type of liposomes as well as mannitol’s weight/molar ratio to the total lipids in the formulation.

### Example 2: Dry Powder Compositions of AmBisome®

Materials and Methods: AmBisome® (Astellas Pharma Inc.) is an FDA-approved liposomal drug delivery system of amphotericin B for intravenous administration. The liposomes are consisting of hydrogenated soy phosphatidylcholine, cholesterol and distearoylphosphatidylglycerol at weight ratio of 4:1:1.6, respectively. AmBisome® is a lyophilized powder that should be reconstituted in 12 mL of sterile water to yield a 12.5 mL dispersion containing 4 mg/mL amphotericin B, 27.92 mg/mL total lipids as well as 72 mg/mL sucrose. Nevertheless, the dispersion should be diluted with 5% dextrose solution to a final amphotericin B concentration of 1-2 mg/mL before administration (I.A.P.U.).

For the preparation of AmBisome® dry powders using TFFD technology, the original AmBisome® powder was reconstituted in 12 mL of sterile water so that the total sucrose to lipid ratio is about 2.5:1 w/w, respectively. The effect of higher sucrose to total lipid ratios on the particle size distribution of AmBisome® after TFFD and reconstitution was also investigated. Additional sucrose was added to the reconstituted AmBisome® dispersion to reach at sucrose to total lipid ratios of 5:1 and 16:1 w/w, respectively. Then, 125 µL of each dispersion was dropped onto a cryogenically cooled surface to rapidly form frozen thin films. The frozen thin films were subsequently lyophilized using the same drying cycles described in Example 1. They can be stored in a -80° C. freezer until lyophilization.

DLS using a Malvern Zeta Sizer Nano ZS (MA, US) was employed to determine the particle size distribution of liquid liposome dispersions (i.e., before TFFD) and rehydrated dry powders prepared by TFFD. The aerodynamic properties of TFFD-processed dry powder of original AmBisome® formulation (i.e., total sucrose to lipid ratio is about 2.5:1 w/w, respectively) were determined using a Next Generation Pharmaceutical Impactor (NGI) (MSP Corp, Shoreview, MN, USA) connected to a High-Capacity Pump (model HCP5, Copley Scientific, Nottingham, UK) and a Critical Flow Controller (model TPK 2000, Copley Scientific, Nottingham, UK). A high-resistance Plastiape® RS00 inhaler (Plastiape S.p.A, Osnago, Italy) containing size #3 hydroxypropyl methylcellulose capsules was attached to a USP induction port by a molded silicon adapter, and the powder was dispersed to the NGI at the flow rate of 60 L/min for 4 s per each actuation, providing a 4 kPa pressure drop across the device. The deposited powders from the capsule, inhaler device, adapter, induction port, stages 1-7, and the micro-orifice collector (MOC) were collected by diluting with a mixture of dimethyl sulfoxide (DMSO)/methanol (20:80 v/v). The amphotericin B content in the deposited powders was determined colorimetrically at a wavelength of 408 nm. Copley Inhaler Testing Data Analysis Software (CITDAS) Version 3.10 (Copley Scientific, Nottingham, UK) was used to calculate the mass median aerodynamic diameter (MMAD), the geometric standard deviation (GSD) and the fine particle fraction (FPF).

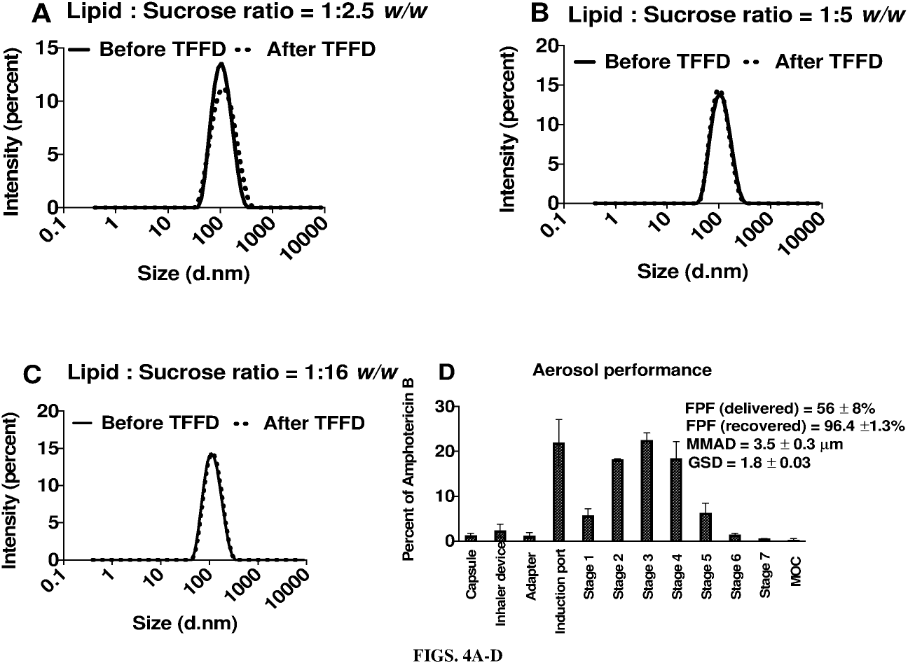

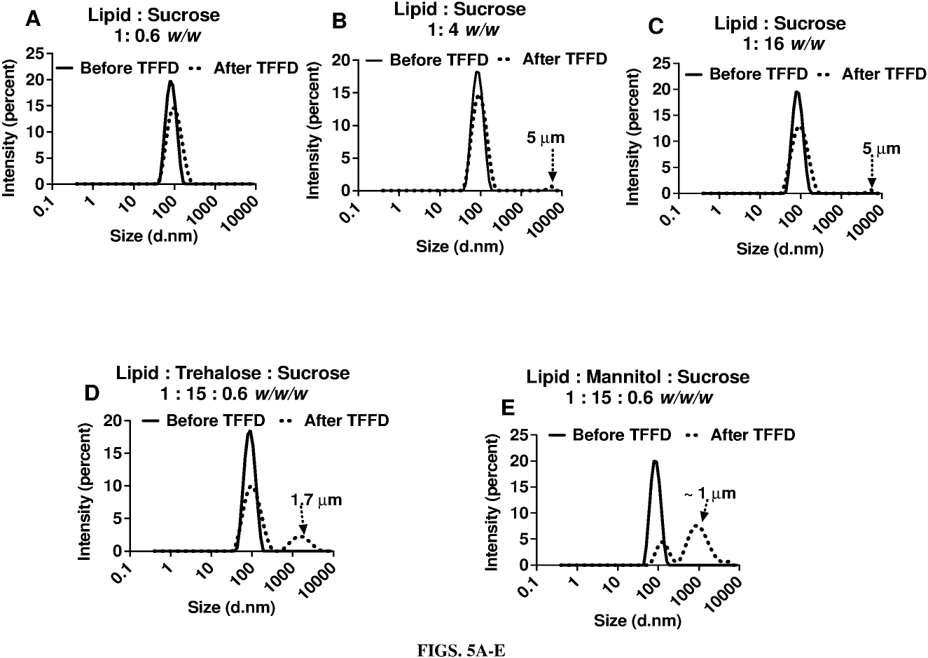

Results: As shown in Example 1, the TFFD technology was proven effective in converting different types of drug-free liposomes (i.e., neutral, anionic and cationic) that are consisting of two types of lipids (i.e., DOPC, DOPA or DOTAP and cholesterol) to dry powders while maintain their particle size distribution after reconstitution. Then, the inventors investigated the applicability of TFFD technology for converting liposomes that have more complexed structure (i.e., consisting of three or more lipids) and loaded with a small molecule active pharmaceutical ingredient (API) to dry powders while maintaining their particle size distribution after reconstitution. AmBisome® liposomal formulation was selected because it is loaded with amphotericin B, an antifungal drug, that holds a great potential for local delivery to the lung. Formulation of amphotericin B liposomes as dry powder for inhalation can further decrease the drug’s systemic side effects (e.g., nephrotoxicity) and enhance the liposome stability. As depicted in FIG. 4A, sucrose, at a concentration already present in AmBisome® liposomal formulation (i.e., 72 mg/mL or sucrose: total lipid ratio of 2.5:1 w/w) was an efficient cryoprotectant that can maintain the liposomes’ particle size distribution after TFFD and reconstitution. Increasing the sucrose to total lipid ratio to 1:5 w/w or 1:16 w/w even better protected the liposomes against freezing and/or drying-induced stresses and thus the particle size distribution was better maintained (FIGS. 4B-C). Importantly, TFFD-processed powder of AmBisome® showed good aerosol performance properties (i.e., delivered FPF of 56 ± 8% and MMAD of 3.5 ± 0.3 µm) without adding additional excipients (e.g., hydrophobic amino acids) although the sucrose content of AmBisome® formulation is relatively high (i.e., 72 mg/mL). Sucrose is a hygroscopic sugar that can increase the moisture uptake and thus decrease the powders’ aerosol performance. TFFD-processed powders are porous and have large surface areas and low densities. Thus, they can reach deep lung after oral inhalation (AboulFotouh et al., 2020). In conclusion, TFFD technology can be utilized to converted liquid liposomal dispersions into inhalable dry powders while maintaining their mean particle size after reconstitution. Thus, this technology possesses a great potential for local lung delivery of antifungal, antibacterial and anticancer agents for treatment of various pulmonary conditions (e.g., cystic fibrosis and Non-Small Cell Lung Cancer) at low drug doses and minimal systemic side effects.

### Example 3: Dry Powder Compositions of Doxil®

Material and Methods: Doxil® (ALZA Corporation, a Johnson & Johnson company) is an FDA-approved liposome injection of doxorubicin HCl for intravenous infusion. It is indicated for the treatment of ovarian cancer, AIDS-related Kaposi’s Sarcoma and Multiple Myeloma. The STEALTH® (ALZA Corporation) liposomes of Doxil® is consisting of N-(carbonyl-methoxy polyethylene glycol 2000)-1,2-distearoyl-sn-glycero- 3-phosphoethanolamine sodium salt (MPEG-DSPE), fully hydrogenated soy phosphatidylcholine and cholesterol at a weight ratio of 1:3:1 w/w/w, respectively. The STEALTH® liposome dispersion also comprises sucrose at a concentration of 10 mg/mL (i.e., sucrose to total lipid ratio of 0.6:1 w/w, respectively) for tonicity adjustment (Nordström et al., 2021).

Firstly, the possibility of converting the STEALTH® liposome dispersion to dry powder using TFFD without adding more sucrose was investigated. The STEALTH® liposome dispersion comprises sucrose at a sucrose to total lipid ratio of 0.6:1 w/w, respectively. The effect of higher sucrose to total lipid ratios on the particle size distribution of the STEALTH® liposomes after TFFD and reconstitution was also investigated. Additional sucrose was added to the liposome dispersion to reach total sucrose to total lipid ratios of 4:1 w/w and 16:1 w/w, respectively. Other cryoprotectants (i.e., trehalose and mannitol) were also investigated at total sucrose to trehalose or mannitol to lipid ratio of 0.6:15:1 w/w/w, respectively. For all formulations, a total volume of 125 µL was dropped onto a cryogenically cooled surface to form frozen thin films rapidly which were then lyophilized as described in Example 1.

Results: In this example the applicability of TFFD technology to process liposomes with more complexed membrane structure to dry powders without significantly affecting their particle size distribution was investigated. The carriers of Doxil® are STEALTH® liposomes in which polyethylene glycol (PEG) polymer is anchored in the liposomal membrane via the cross-linked lipid MPEG-DSPE. The presence of PEG on the liposome surface extends the liposomes’ blood circulation time (Immordino et al., 2006). As depicted in FIG. 5A, the STEALTH® liposome dispersion can be processed into dry powder while maintaining the liposomes’ particle size distribution without adding more cryoprotectant (i.e., sucrose). When the sucrose to total lipid weight ratio was increased to either 4:1 w/w or 16:1 w/w, respectively, few aggregates (~1.5 % intensity) having mean particle size of ∼ 5 µm were detected (FIGS. 5B-C). Trehalose and mannitol at the screened ratio resulted in a significant liposome membrane fusion (FIGS. 5D-E). The aggregation was more pronounced when mannitol was incorporated in the formulation compared to trehalose. In conclusion, TFFD technology can convert liposomes with complexed membrane structures (e.g., STEALTH® liposomes) to dry powders without a significant effect on their particle size distribution after reconstitution at low cryoprotectant to lipid ratios.

### Example 4: Dry Powder Compositions of CpG ODNs-Loaded Liposomes

Materials and methods: DOTAP-cholesterol cationic liposomes were prepared as described in Example 1. Then the liposomes were mixed with Cytosine-Phosphate-Guanine Oligodeoxynucleotides (CpG ODNs, (ODN 1826, InvivoGen)) at a weight ratio of 1:1 at room temperature for 15 min. Sucrose, trehalose or mannitol was incorporated in the formulation as a cryoprotectant at a cryoprotectant to total lipid ratio of 250:1 w/w, 500:1 w/w and 1000:1 w/w, respectively. To prepare dry powders of CpG ODNs-loaded liposomes, 125 µL of each formulation was dropped onto a cryogenically cooled surface to form frozen thin films rapidly which were then lyophilized as described in Example 1.

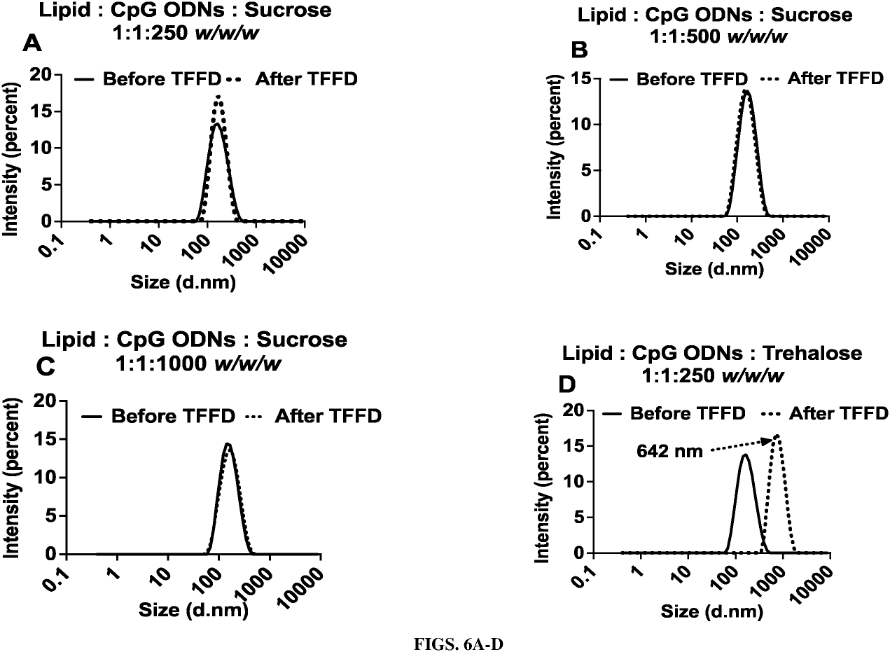

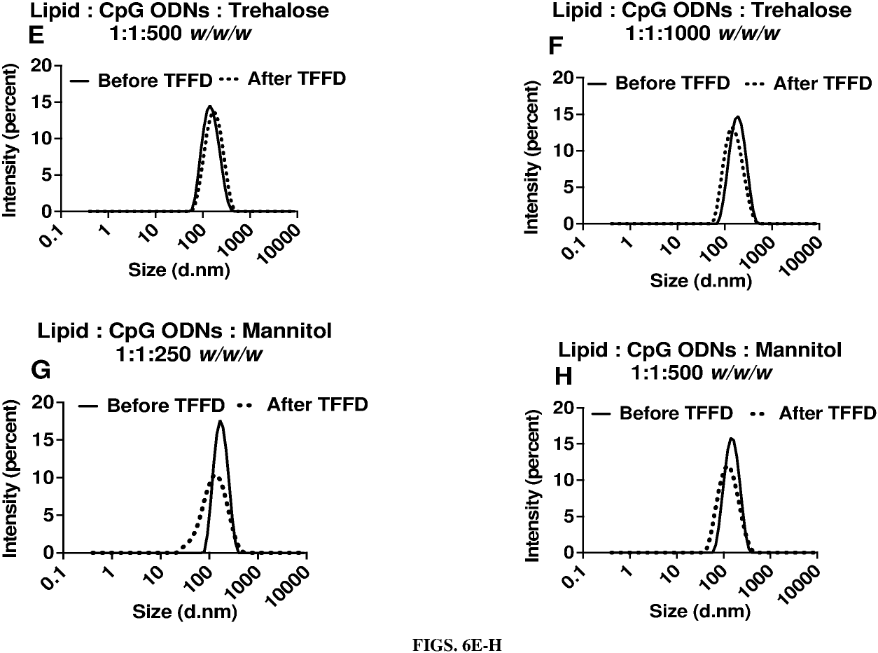

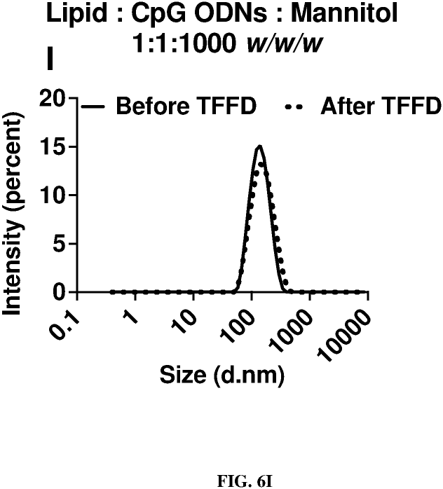

Results: In this example the applicability of TFFD technology to process liposomes loaded with macromolecules (i.e., ODNs) to dry powders without significantly affecting their particle size distribution after reconstitution was investigated. CpG ODNs were selected as an example of macromolecules that can be loaded to the cationic liposomes. They are short single-stranded deoxyribonucleic acid that trigger activation of the downstream Toll-like receptor 9 (TLR9) and secretion of proinflammatory cytokines. Hence, CpG ODNs are promising vaccine adjuvants and immunostimultor (Adamus & Kortylewski, 2018). CpG ODNs possess a great promise for local lung delivery to stimulate mucosal immune response (i.e., vaccine adjuvant) or treatment of non-small-cell lung cancer. Particularly, formulation of inhalable dry powders of CpG ODNs can both enhance their stability and reduce their systemic toxicity. Surprisingly, mannitol was as effective as sucrose and more effective than trehalose as a cryoprotectant. As shown in FIGS. 6A-C and FIGS. 6G-I, both sucrose and mannitol were effective as cryoprotectants that can maintain the particle size distribution of CpG ODNs-loaded liposomes after TFFD and reconstitution at all screened ratios. Trehalose was effective as a cryoprotectant when it was incorporated in the formulation at trehalose to total lipid ratio of 500:1 w/w or 1000:1 w/w. Overall, TFFD technology can converted macromolecule-loaded liposomes to dry powders while maintaining the liposomes’ particle size after reconstitution.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### V. References

The following references, to the extent that they provide exemplary procedural or other details supplementary to those set forth herein, are specifically incorporated herein by reference.

AboulFotouh, et al., “Amorphous solid dispersion dry powder for pulmonary drug delivery: Advantages and challenges,” Int J Pharm, 587 (2020) 119711.

AboulFotouh et al., “Next-Generation COVID-19 Vaccines Should Take Efficiency of Distribution into Consideration.” AAPS PharmSciTech, 2021. 22(3): p. 126.

AboulFotouh et al., “Development of (Inhalable) Dry Powder Formulations of AS01(B)-Containing Vaccines Using Thin-Film Freeze-Drying”. Int J Pharm, 2022: p. 121825.

Barenholz, “Liposome application: problems and prospects,” Current Opinion in Colloid & Interface Science, 6 (2001) 66-77.

Boyce et al., “Safety and immunogenicity of adjuvanted and unadjuvanted subunit influenza vaccines administered intranasally to healthy adults”. Vaccine, 2000. 19(2-3): p. 217-26.

Drulis-Kawa and Dorotkiewicz-Jach, “Liposomes as delivery systems for antibiotics,” Int J Pharm, 387 (2010) 187-198.

Engstrom et al., “Formation of stable submicron protein particles by thin film freezing,” Pharm Res, 25 (2008) 1334-1346.

Franze et al., Lyophilization of Liposomal Formulations: Still Necessary, Still Challenging, Pharmaceutics, 10 (2018).

Hufnagel, et al., “The Development of Thin-film Freezing and Its Application to Improve Delivery of Biologics as Dry Powder Aerosols,” KONA Powder and Particle Journal, advpub (2022).

Immordino et al., “Stealth liposomes: review of the basic science, rationale, and clinical applications, existing and potential,” Int J Nanomedicine, 1 (2006) 297-315.

Adamus & Kortylewski, “The revival of CpG oligonucleotide-based cancer immunotherapies”, Contemp Oncol (Pozn), 22 (2018) 56-60.

Ingvarsson et al., “Stabilization of liposomes during drying,” Expert Opin Drug Deliv, 8 (2011) 375-388.

I.A.P.U. AmBisome [package insert]. Northbrook, Inc.

Mandon et al., “Novel calixarene-based surfactant enables low dose split inactivated vaccine protection against influenza infection.” Vaccine, 2020. 38(2): p. 278-287.

Nordström et al., “Quantitative Cryo-TEM Reveals New Structural Details of Doxil-Like PEGylated Liposomal Doxorubicin Formulation”, Pharmaceutics, 13 (2021).

Ren et al., “Intranasal Immunization Using Mannatide as a Novel Adjuvant for an Inactivated Influenza Vaccine and Its Adjuvant Effect Compared with MF59.” PLoS One, 2017. 12(1): p. e0169501.

Sahakijpijarn et al., “Development of Remdesivir as a Dry Powder for Inhalation by Thin Film Freezing,” Pharmaceutics, 12 (2020).

Sahakijpijarn et al., “In vivo pharmacokinetic study of remdesivir dry powder for inhalation in hamsters,” International Journal of Pharmaceutics: X, 3 (2021) 100073.

Salade et al., “How to characterize a nasal product. The state of the art of in vitro and ex vivo specific methods.” Int J Pharm, 2019. 561: p. 47-65.

Skwarczynski, M. and I. Toth, “Non-invasive mucosal vaccine delivery: advantages, challenges and the future.” Expert Opin Drug Deliv, 2020. 17(4): p. 435-437.

Yanasarn et al., “Negatively charged liposomes show potent adjuvant activity when simply admixed with protein antigens”, Mol Pharm, 8 (2011) 1174-1185.

Yu, et al., “Inhalable liposomal powder formulations for co-delivery of synergistic ciprofloxacin and colistin against multi-drug resistant gram-negative lung infections,” Int J Pharm, 575 (2020) 118915.

Wang et al., “Effect of processing parameters on the physicochemical and aerodynamic properties of respirable brittle matrix powders,” Journal of Drug Delivery Science and Technology, 24 (2014) 390-396.

www.fda.gov. “Fluzone Quadrivalent Package Insert. The Food and Drug Administration,” accessed Feb. 3, 2022, https://www.fda.gov/media/119856/download.

Xu et al., “Immunogenicity of Antigen Adjuvanted with AS04 and Its Deposition in the Upper Respiratory Tract after Intranasal Administration.” Mol Pharm, 2020. 17(9): p. 3259-3269.

Zhang et al., “Novel formulations and drug delivery systems to administer biological solids.” Adv Drug Deliv Rev, 2021. 172: p. 183-210.

1-15. (canceled)

16. A method for preparing a thin film liposomal composition comprising:
applying a liquid liposomal composition and a sugar or a sugar alcohol to a freezing surface; and
allowing said liquid liposomal composition to disperse and freeze on said freezing surface thereby forming a thin film.

17. The method of claim 16, wherein thin film liposomal composition further comprises a drug or biologic agent.

18. The method of claim 16, wherein said liquid liposomal composition comprises dioleoyl phosphatidylcholine and cholesterol.

19. The method of claim 16, wherein said thin film has a particle size distribution upon reconstitution within about 10-50% or about 30% of the range of the liquid liposomal composition.

20. The method of claim 16, wherein said sugar or sugar alcohol is present at about a lipid to sugar and/or sugar alcohol ratio of about 1:0.6 to 1:30 w/w, respectively or about 1:250 to 1:1000 w/w, respectively.

21. The method of claim 16, wherein the sugar or sugar alcohol is mannitol, sucrose and/or trehalose.

22. The method of claim 16, wherein said thin film comprises less than about 5% water.

23. The method of claim 16, wherein said thin film further comprises an excipient.

24. The method of claim 23, wherein said excipient is a salt, a buffer, a detergent, a polymer, an amino acid, a second sugar or a preservative.

25. The method of claim 24, wherein said excipient is disodium edetate, sodium chloride, sodium citrate, sodium succinate, sodium hydroxide, sodium glucoheptonate, sodium acetyltryptophanate, sodium bicarbonate, sodium caprylate, sodium pertechnetate, sodium acetate, sodium dodecyl sulfate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, agarose, sorbitol, maltose, trehalose, surfactants, polysorbate 80, polysorbate 20, poloxamer 188, sorbitan monooleate, triton n101, m-cresol, benyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol, glyclyglycine, thimerosal, parabens, gelatin, Formaldehyde, Dulbecco’—s modified eagles medium, hydrocortisone, neomycin, Von Willebrand factor, gluteraldehyde, benzethonium chloride, white petroleum, p-aminopheyl-p-anisate, monosodium glutamate, beta-propiolactone, acetate, citrate, glutamate, glycinate, histidine, Lactate, Maleate, phosphate, succinate, tartrate, tris, carbomer 1342 (copolymer of acrylic acid and a long chain alkyl methacrylate cross-linked with allyl ethers of pentaerythritol), glucose star polymer, silicone polymer, polydimethylsiloxane, polyethylene glycol, polyvinylpyrrolidone, carboxymethylcellulose, poly(glycolic acid), poly(lactic-co-glycolic acid), polylactic acid, dextran 40, or poloxamer.

26. The method of claim 23, comprising from about 60% to about 95% w/w of said excipient.

27. The method of claim 16, wherein said liquid liposomal composition is exposed to said freezing surface from about 50 milliseconds to about 5 seconds.

28. The method of claim 16, wherein exposure comprises spraying or dripping droplets of said liquid liposomal composition.

29. The method of claim 28, wherein the freezing surface temperature is about -180° C. to about 0° C., the diameters of the droplets are about 2-5 millimeters, and the droplets are dropped from a distance about 2 cm to 10 cm from the freezing surface.

30. The method of claim 28, further comprising contacting the droplets with a freezing surface having a temperature differential of at least about 30° C. between the droplets and the surface.

31. The method of claim 30, wherein the freezing rate of said droplets is between about 10 K/second and about 10³ K/second.

32. The method of claim 31, further comprising removing the solvent from the thin film to form a dry composition optionally by lyophilization.

33. (canceled)

34. The method of claim 32, further comprising solvating said dry liposomal composition, thereby forming a reconstituted liquid composition.

35. The method of claim 34, wherein the liquid liposomal composition further comprises a drug or biologic agent, wherein the drug or biological agent is an antifungal, an antibacterial, a cancer chemotherapeutical agent, a protein (e.g., enzyme, monoclonal antibody), or nucleic acid.

36. (canceled)

37. The method of claim 16, wherein the thin film liposomal composition is formulated as an aerosol/for inhalation.

38-49. (canceled)
